(19) United States
(12) Patent Application Publication
(10) Pub. No.: US 2006/0111727 A1
(43) Pub. Date: May 25, 2006

(54) Device for delivering a surgical implant

(76) Inventors: Martin Pfleiderer, Leeds (GB); Alec Birkbeck, Leeds (GB)

Correspondence Address: Paul J Maginot;Maginot Moore & Beck, Bank One Center Tower, 111 Monument Circle Suite 3000, Indianapolis, IN, 46204-5115, US

(21) Appl. No.: 10/527,149

(22) PCT Filed: Sep. 16, 2003
(86) PCT No.: PCT/GB03/03987

§ 371 Date: Oct. 20, 2005

(30) Foreign Application Priority Data
Sep. 17, 2002 (GB) 0221527.5

(51) Int. Cl. A61B 17/90; A61F 2/46; A61F 2/44

(52) U.S. Cl. 606/86; 623/17.11

(57) ABSTRACT

A device (10) for delivering a surgical implant (14) comprises a guide track (11) for guiding the movement of a spinal implant (14) to an implantation site. An implant carrier (12) engages the implant in the guide track and can move along the guide track in order to deliver the implant to the implantation site, the carrier (12) including a connector (15) which can be displaced between a connected position in which the implant is connected to the carrier to move with it in the guide track, and a disconnected position in which the implant can be separated from the carrier. A driving device (13) can engage the implant carrier to move the carrier (12) along the track. A formation can cause the connector (15) on the implant carrier (12) to be displaced from the connected to the disconnected position when the carrier reaches a pre-determined position.

This invention relates to a device for delivering a surgical implant.

Interbody spinal implants are fitted between vertebrae to stabilise the vertebrae. An implant can be implanted as a disk replacement. An implant can be used in fusion of the vertebrae.

A known spinal implant is disclosed in WO-A-01/06962. It comprises a cage made from a shape memory alloy. It is introduced into the space between two vertebrae through a minimal access opening (posterior approach).

Ease of manipulation of the implant is a very important requirement; also a highly desirable feature of an implantation device is to facilitate easy cleansing and sterilisation after use, given that body fluids can easily enter the interior of any device used for spinal implantation.

In one aspect, the present invention provides a device for delivering a surgical implant, which comprises:

a guide track for guiding the movement of a spinal implant to an implantation site of a patient;

an implant carrier for engaging an implant in the guide track and which can move along the guide track in order to deliver the implant to the implantation site, the carrier including a displaceable connector which can be displaced between a connected position in which the implant is connected to the carrier to move with it in the guide track, and a disconnected position in which the implant can be separated from the carrier;

a driving device which can engage the implant carrier to move the carrier and an implant which engages the carrier along the track; and

a formation which causes the displaceable connector on the implant carrier to be displaced from the connected position to the disconnected position when the carrier reaches a pre-determined position relative to the guide track, to allow the implant to be released from the device for implantation.

The invention therefore enables easy guidance of an implant to an implantation site and, upon release of the implant, the surgeon may then manually complete the implantation. When, as is preferred, the implant is formed from a shape memory alloy, it can revert from a deformed configuration in which it is held within the guide track to an in-use configuration, especially involving straightening of the implant.

Preferably, the connector is pivotably connected to the implant carrier. The connector can then be displaced from the connected position to the disconnected position by pivoting. Other displacements are envisaged. For example, the connector might be displaced sliding or translating.

Preferably, the driving device is a manually operative device and conveniently advancing and reversing movement of the elongate carrier can be carried out by manipulation of finger-operated levers.

Preferably, the formation comprises a ramp. The ramp can be inclined to the guide track.

Preferably, the formation comprises an recess into which the connector can be displaced, for example by pivoting. Preferably the opening (for example with an associated ramp) is provided in the bottom face of the guide track so that the formation can drop into the opening when positioned adjacent thereto.

In a preferred arrangement, the implant carrier takes the form of a toothed rack, and the driving device may include a ratchet-type of drive pin or the like to engage intermittently with the rack, and thereby cause incremental advance or return movement of the rack as required. However, other drive connections may be provided between the driving device and the toothed rack, including a drive pinion.

Preferably, the guide track is in the form of a channel in which the implant can slide. The channel is preferably at least partially enclosed so that the implant can be discharged from the channel at or towards one end, and not through the top or bottom of the track. The guide track can be generally C-shaped when viewed in cross-section, especially where the open side of the “C” is directed upwardly so that the re-entrant portions prevent the implant from being removed from within the track through the top thereof.

The track should be dimensioned so that the implant is a sliding fit within it. The track will generally have a constant cross-section along its length. When the implant is a spinal implant to be fitted between two vertebrae, its depth will generally in the range 4 to 8 mm. Accordingly, the width of the track is preferably at least about 4 mm, more preferably at least about 8 mm. The width of the track will often be not more than about 15 mm, especially not more than about 12 mm. The thickness of a spinal implant will generally be in the range 0.5 to 1.5 mm. The depth of the track is preferably at least about 0.5 mm, more preferably at least about 1.0 mm, for example at least about 1.5 mm.

The implant and the connector can be connected by means of interfitting plug and recess. The recess will often have a reentrant shape so that the plug can only be fitted into it by sliding it in a direction which is not aligned with the direction in which the implant moves in the guide track, often generally perpendicular to that direction. Suitable reentrant shapes are often referred to as dove-tail shapes, although it will be understood that the recess which is used in the present invention can be rounded or angular. Accordingly, it can be preferred for one of the implant and the connector on the carrier to have a dove-tail shape at its end, and the other to have a recess in which the dove-tail shape can be received.

Preferably, the driving device has a housing in which the driving components are housed, and the housing may be separable into at least two separate portions to allow easy access to the interior for the purposes of cleaning/sterilisation of the internal components, and the interior of the housing, after surgery. Preferably, the driving device includes at least one driver lever for causing movement of the implant carrier in the guide track.

Embodiments of devices according to the invention will now be described, by way of example, with reference to the accompanying drawings, in which:

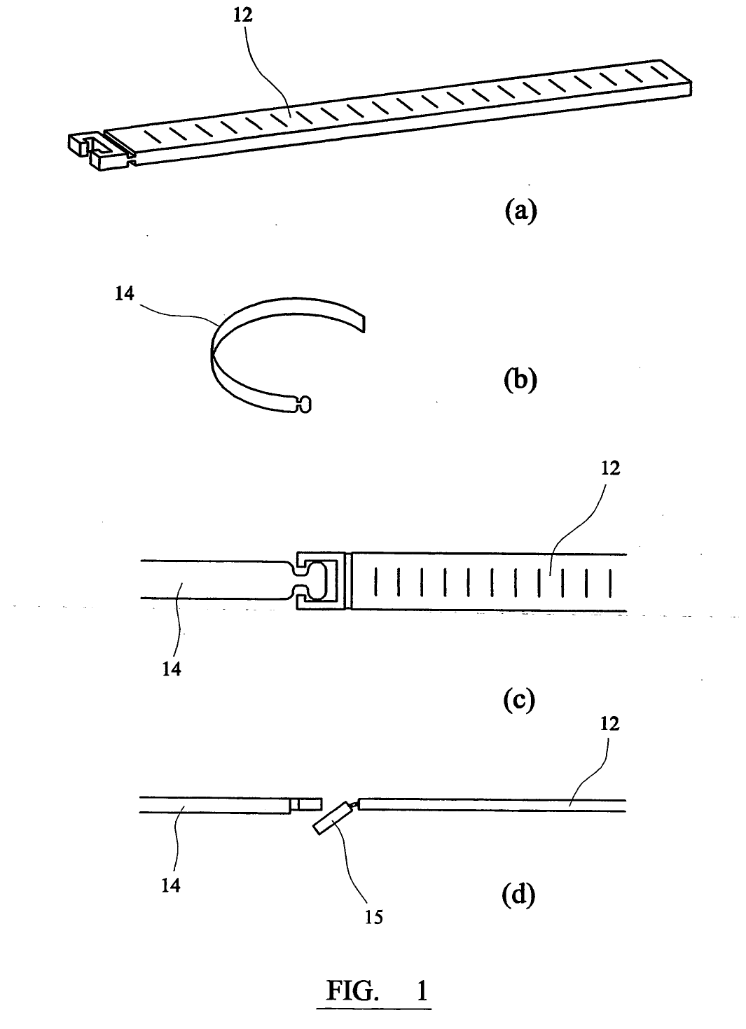

FIGS. 1a, b, c and d are separate views of, respectively, the implant carrier, the implant, the implant coupled to one end of the implant carrier, and a pivoted release arrangement at one end of the implant carrier for effecting release of the carrier after the latter has been delivered to the implantation site.

FIG. 1 is a perspective view from one side of a manually operative driving device, guide track, and implant carrier, in non-assembled form, and forming a spinal implantation device according to the invention for use in delivering a spinal implant to an implantation site of a patient;

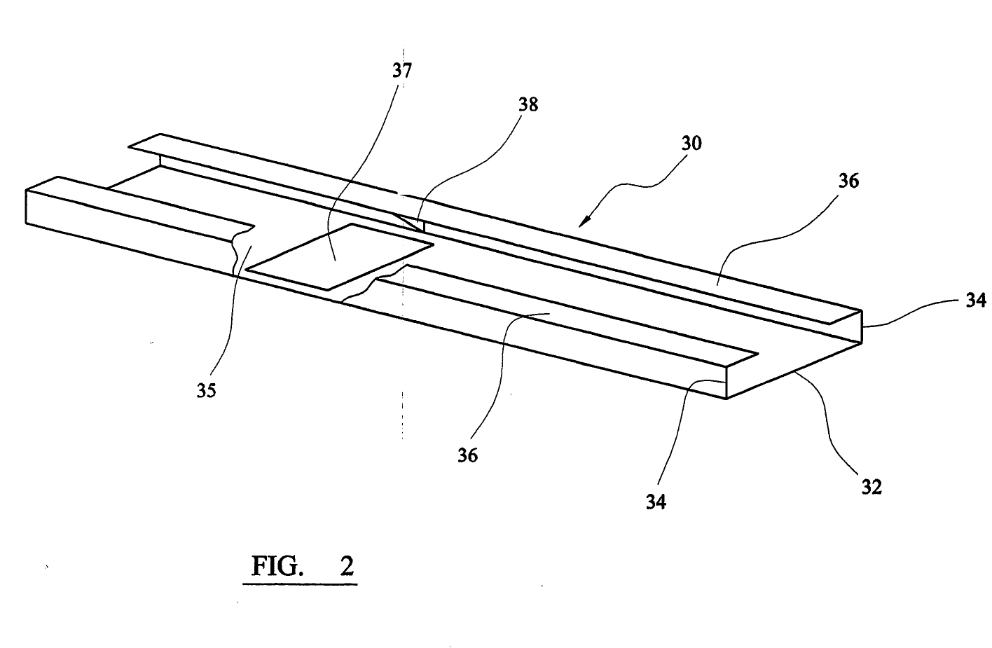

FIG. 2 is a perspective view, from an opposite side of the housing of the driving device, showing manually operative levers for applying advancing and reversing movement to the implant carrier;

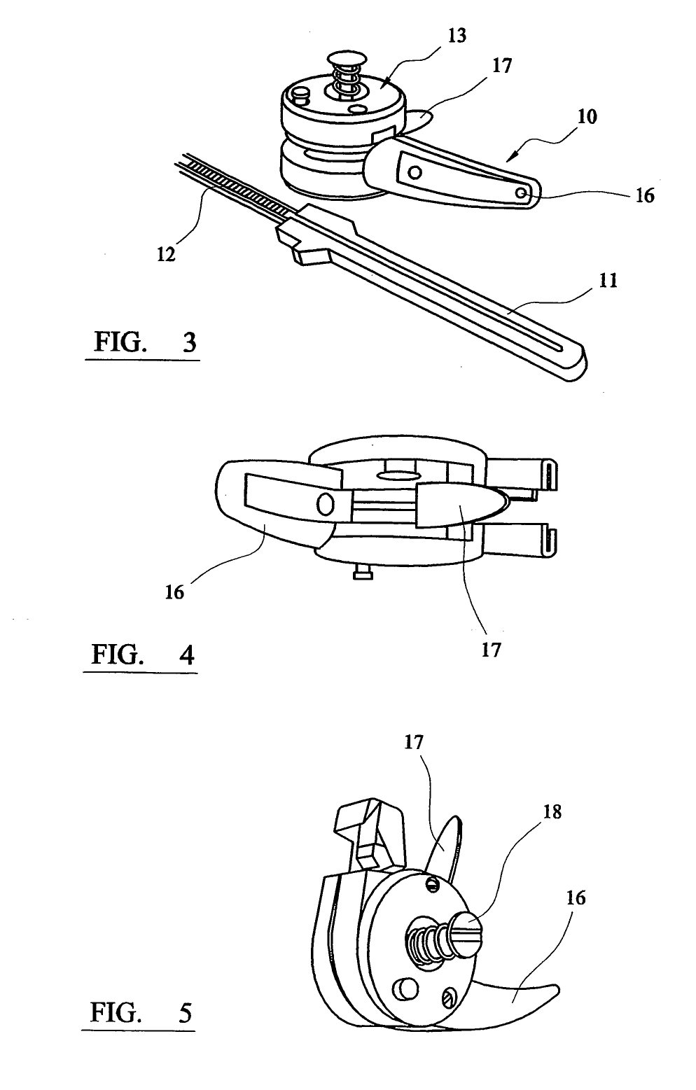

FIG. 3 is a perspective illustration, partly in plan view, showing in more detail, the mounting on the driving device on which the guide track and implant carrier can be mounted; and

Referring now to the drawings, a spinal implantation device according to the invention is designated generally by reference 10 and comprises a guide track 11 for guiding the movement of the spinal implant to an implantation site of a patient, and an elongate implant carrier 12 which is moveable lengthwise of the guide track 11 in order to deliver the implant to the implantation site, and a driving device 13 which is engageable with the elongate carrier 12 and operative to apply indexing movement to the carrier.

FIG. 1 shows separate views of the implant carrier 12, and an implant 14, and how they work together in order to deliver the implant to the required patient site and then effect release of the implant for final completion of the implantation process.

FIG. 1c shows the implant 14 coupled to a leading end of the implant carrier 12 and FIG. 1d shows the implant after release from the carrier, when it has been delivered to the implantation site. There is shown schematically a pivotable element 15, which in the illustrated arrangement is connected to the leading end of the carrier 12 and which engages with the implant 14, when the latter has reached the implantation site, so as to release the implant from the carrier 12 and allow completion of the implantation.

The invention therefore provides for easy guidance of the implant 14 to the implantation site, and upon release of the implant, the surgeon may then manually complete the implantation. When, as is preferred, and shown in FIG. 1b, the implant is a curved shape memory cage, this may easily be manipulated so as to complete implantation.

FIG. 2 shows the guide track 30 which has a reentrant shape when viewed in cross section along its length defined by a base 32, side walls 34 and top walls 36 which cover only the edges of the base 32. The width of the track is such that the implant carrier 12 is a close sliding fit between the side walls 34, and between the base 32 and the top walls 36. A part 35 of one of the side walls has been cut away in FIG. 2 to enable internal features of the track to be seen.

The base 32 has an aperture 37 formed in it which extends across most of the width of the track and is large enough for the pivotable element 15 on the implant carrier to be displaced into. The aperture can be open, or can be in the form of a recess which is closed at its base. This can be preferred in order to control the extent of the displacement of the pivotable element.

Each of the top walls 36 has a ramp 38 formed in it, in the surface which faces towards the base. The ramp is located at about one of the edges of the aperture. It has an inclined surface facing towards the base of the track.

The width of the implant is less than the distance between the ramps 38 (measured across the guide track) so that the implant can slide along the track past the ramps. The width of the pivotable element 15 is greater than the distance between the ramps so that the pivotable element is acted on by the ramps as it passes under the ramps. These width features are apparent from FIG. 1c.

The action of the ramps on the pivotable element displaces the element pivotally into the aperture 37. The nature of the connection between the pivotable element is such that the implant is released from the implant carrier as a result of this displacement (as shown in FIG. 1d). After the implant has been released from the carrier, the carrier can withdrawn along the guide track. The pivotable element 15 then resumes its aligned configuration which it adopted before displacement by the ramps.

Referring to FIGS. 3 to 5, this shows some features of the construction and operation of a manually operated driving device 13 in which advancing and reversing movement of the elongate carrier 12 is carried out by manual manipulation of finger operated levers 16 and 17.

The implant carrier 12 preferably takes the form of a toothed rack, as shown, and the driving device 13 may include a ratchet-type of drive pin (not shown in detail) or the like, to engage intermittently with the rack, and thereby cause incremental advance or return movement of the rack as required. Suitable ratchet drive mechanisms can be devised based on conventional mechanisms as used in various applications. Other drive connections may be provided, to transmit linear reciprocating movement to the rack 12, including a drive pinion.

The driving device 13 has a two part housing in which the driving components are housed, and which may be separated, by operation of a push button 18, to allow easy access to the interior for the purposes of cleansing/sterilising the internal components after surgery and also the interior of the housing.

1. A device for delivering a surgical implant, which comprises:
a guide track for guiding the movement of a spinal implant to an implantation site of a patient;
an implant carrier for engaging an implant in the guide track and which can move along the guide track in order to deliver the implant to the implantation site, the carrier including a displaceable connector which can be displaced between a connected position in which the implant is connected to the carrier to move with it in the guide track, and a disconnected position in which the implant can be separated from the carrier;
a driving device which can engage the implant carrier to move the carrier and an implant which engages the carrier along the track; and
a formation which causes the displaceable connector on the implant carrier to be displaced from the connected position to the disconnected position when the carrier reaches a pre-determined position relative to the guide track, to allow the implant to be released from the device for implantation.

2. A device as claimed in claim 1, in which the formation is located at or towards the end of the guide track from which the implant is delivered.

3. A device as claimed in claim 1, in which the connector is pivotably connected to the implant carrier.

4. A device as claimed in claim 1, in which the guide track includes a recess in it into which the connector can be displaced to allow it to be disconnected from the implant.

5. A device as claimed in claim 1, in which the formation comprises a ramp.

6. A device as claimed in claim 1, in which the driving device is manually operated.

7. A device as claimed in claim 1, in which the implant carrier comprises a toothed rack, and the driving device includes a drive pin or the like to engage with the rack.

8. A device as claimed in claim 1, in which the driving device includes a housing in which driving components are housed.

9. A device as claimed in claim 7, in which the housing is separable into at least two separate portions to allow access to its interior for cleaning purposes.

10. A device as claimed in claim 1, in which the driving device includes at least one driver lever for causing movement of the implant carrier in the guide track.

11. An assembly which comprises a device for delivering a surgical implant as claimed in claim 1, and a surgical implant fitted within the guide track.

12. An assembly as claimed in claim 11, in which the surgical implant is formed from a shape memory alloy.

13. An assembly as claimed in claim 11, in which the surgical implant can revert from a deformed configuration which it adopts while in the guide track and an in-use configuration which it adopts once discharged from the guide track.

14. An assembly as claimed in claim 13, in which the configuration of the implant when in use is curved and the configuration of the implant when deformed for implantation is essentially straight.

15. An assembly as claimed in claim 11, in which the surgical implant is a spinal implant to be fitted between two vertebrae.

16. An assembly as claimed in claim 11, in which one of the implant and the connector on the carrier has a dove-tail shape at its end, and the other has a recess in which the dove-tail shape can be received.
